(19) United States
(12) Patent Application Publication
(10) Pub. No.: US 2018/0242916 A1
(43) Pub. Date: Aug. 30, 2018

(54) ELECTROENCEPHALOGRAM MONITORING SYSTEM AND METHOD OF USE OF THE SAME

(71) Applicant: THE GENERAL HOSPITAL CORPORATION, Boston, MA (US)

(72) Inventors: Patrick L. Purdon, Somerville, MA (US); Michael J. Prerau, Newton, MA (US); Nathaniel M. Sims, Boston, MA (US); Matthew Hickcox, Bolton, MA (US)

(21) Appl. No.: 15/756,933

(22) PCT Filed: Sep. 2, 2016
(86) PCT No.: PCT/US16/50230

§ 371 Date: Mar. 1, 2018

Related U.S. Application Data
(60) Provisional application No. 62/213,232, filed on Sep. 2, 2015.

(51) Int. Cl. A61B 5/00; A61B 5/0478; A61B 5/0492; A61B 5/0408; A61B 5/04; A61B 5/0205

(52) U.S. Cl. CPC A61B 5/7203; A61B 5/0533; A61B 5/0478; A61B 5/0492; A61B 5/04085; A61B 5/04017; A61B 5/7232; A61B 5/4821; A61B 5/4812; A61B 5/4848; A61B 5/04087; A61B 5/0205; A61B 2562/164; A61B 2562/182; A61B 5/4824; A61B 5/0006

(57) ABSTRACT

A system and method for monitoring a subject are provided. In some aspects, a provided electroencephalogram (“EEG”) system includes an electrode patch assembly configured to attach to a subject's skin, the assembly including a flexible circuit layer having electrical leads configured to acquire EEG signals from the subject, the flexible circuit layer having a shielding layer configured to substantially reduce a coupling of the plurality of electrical leads to external sources of noise, and a holder to which the flexible circuit layer is secured. The system also includes an electronics module removably coupled to the holder and configured to engage electrical contacts on the flexible circuit layer, the module including a front-end module configured to perform an active noise cancellation process on the acquired EEG signals and generate digitized data using noise cancelled signals, and a processor configured to transmit the digitized data using a wireless communication module.

### RELATED APPLICATIONS

This application is based on, claims priority to, and incorporates herein by reference in its entirety, U.S. Provisional Application No. 62/213,232, filed on Sep. 2, 2015, and entitled “Wireless EEG sensors.”

### STATEMENT REGARDING FEDERALLY SPONSORED RESEARCH

This invention was made with government support under Grant No. DP2-OD006454 awarded by the National Institutes of Health. The government has certain rights in the invention.

### BACKGROUND OF THE INVENTION

The present disclosure generally relates to systems and method for patient monitoring and, more particularly, to systems and methods for wireless monitoring of physiological signals.

Electroencephalogram (“EEG”) and other physiological monitoring has become a standard practice being used to diagnose and treat patients in various clinical settings, including operating rooms and intensive care units. For instance, a number of EEG monitoring systems have been developed to help track the level of consciousness of patients receiving general anesthesia or sedation during surgery or other medical procedures, such as a medically induced coma. By analyzing EEG measurements, monitoring systems typically provide feedback to clinicians in the form of partial or amalgamated representations indicating the condition of the patient at any one time.

For instance, traditional monitoring systems have often quantified a patient's depth of anesthesia through a single dimensionless index, such as the so-called Bispectral Index (“BIS”). In particular, the BIS is derived by computing spectral and bispectral features from acquired EEG waveform. The computed features are then provided as input to proprietary algorithms to derive an index between 0 and 100. Decreased BIS values indicate deepening levels of anesthesia or sedation, with 100 corresponding to a fully awake state and 0 corresponding to the most profound state of coma. More recent approaches have identified and implemented other information extracted from acquired EEG data to indicate various states of anesthesia and sedation, including patterns in signal spectra, transient signals, signal coherence and synchrony, to name but a few.

EEG monitors have also been used to help diagnose and treat sleep disorders. In particular, sleep is a natural, restorative, altered state of consciousness common to every living human being. Neurophysiologically, sleep is a continuous, dynamic process involving the complex interaction of cortical and sub-cortical networks within the brain operating on multiple time scales. As such, EEG monitoring naturally provides brain activity information, including correlates of activity from numerous brain regions. Furthermore, EEG data has also been used to study psychological (e.g., schizophrenia, depression, and anxiety) and neurological (e.g., Alzheimer's disease and Parkinson's disease) disorders, which affect millions of people worldwide and are often associated with disrupted sleep dynamics,

Although much progress has been made in identifying key indicators of states of consciousness and sleep, their accuracy relies on the quality and reliability of the acquired EEG signals. However, in realistic clinical situations, such as during surgery, EEG data can be subjected to various sources of noise and distortion. For instance, current monitoring systems include EEG sensors with extended wires, which act as antennas that pick up external noise from equipment and machines as well as spurious noise present in the operating room. Also, long wires can be quite cumbersome for medical staff during surgery, and other medical procedures. In addition, EEG sensors used in current monitoring systems are typically incorporated into headbands or headsets that are either uncomfortable or unsuitable for long term use, or are prone to artifacts or signal interruptions due to poor connectivity to the patient.

Clinical investigations of sleep disorders, and other neurological conditions, have often been limited to EEG monitoring systems in sleep labs or other clinical settings. Notwithstanding that many such clinical EEG systems are not amenable to non-clinical or home applications, they also suffer from similar data noise and distortion, particularly due to movement during sleep. In addition, although wearable consumer devices are increasingly being used for various sleep information applications, data provided by these devices do not have clinically proven reliability or accuracy, and are hence not used for scientific or medical purposes.

In light of the above, there is a need for improved technologies for monitoring physiological signals from patients.

### SUMMARY

The present disclosure provides a novel system and method for monitoring of physiological signals, including wireless electroencephalogram (“EEG”) and other physiological monitoring. As will be described, the provided system and method includes elements and features that overcome drawbacks of present technologies.

In one aspect of the disclosure, an electroencephalogram (“EEG”) monitoring system is provided. The system includes an electrode patch assembly configured to attach to a subject's skin, the electrode patch assembly comprising a flexible circuit layer having a plurality of electrical leads configured to acquire EEG signals from the subject, the flexible circuit layer having a shielding layer configured to substantially reduce a coupling of the plurality of electrical leads to external sources of noise, and a holder to which the flexible circuit layer is secured. The system also includes an electronics module removably coupled to the holder and configured to engage electrical contacts on the flexible circuit layer, the electronics module comprising a front-end module configured to perform an active noise cancellation process on the acquired EEG signals and generate digitized data using noise cancelled signals and a processor configured to transmit the digitized data using a wireless communication module. The system further includes an external device configured to receive and analyze the digitized data transmitted to determine a condition of the subject.

In another aspect of the disclosure, a system for wirelessly monitoring a subject is provided. The system includes an electrode patch assembly configured to attach to a subject's skin, the electrode patch assembly comprising a flexible circuit layer having a plurality of electrical leads configured to acquire physiological signals from the subject, the flexible circuit layer having a shielding layer configured to substantially reduce a coupling of the plurality of electrical leads to external sources of noise, and a holder to which the flexible circuit layer is secured. The system also includes an electronics module removably coupled to the holder and configured to engage electrical contacts on the flexible circuit layer, the electronics module comprising a front-end module configured to generate digitized data using the acquired physiological signals and a processor configured to wirelessly transmit, using a transceiver in the electronics module, the digitized data. The system further includes an external device configured to communicate with the electronics module using a wireless communication protocol to receive the digitized data transmitted.

In yet another aspect of the disclosure, a system for wirelessly monitoring a subject is provided. The system includes an electrode patch assembly configured to attach to a subject's skin, the electrode patch assembly comprising a flexible circuit layer having a plurality of electrical leads configured to acquire physiological signals from the subject, the flexible circuit layer having a shielding layer configured to substantially reduce a coupling of the plurality of electrical leads to external sources of noise, and a holder to which the flexible circuit layer is secured. The system also includes an electronics module removably coupled to the holder and configured to engage electrical contacts on the flexible circuit layer using an electrical coupling, the electronics module comprising a front-end module configured to generate digitized data using the acquired physiological signals and a processor configured to compress and wirelessly transmit the digitized data, using a transceiver in the electronics module.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of a wireless monitoring system, in accordance with aspects of the present disclosure.

FIG. 2A is a perspective view of one embodiment of the wireless monitoring system of FIG. 1.

FIG. 2B is another perspective view of the embodiment of FIG. 2A.

FIG. 2C is yet another perspective view of the embodiment of FIG. 2A.

FIG. 2D is a sectional view of the embodiment of FIG. 2A.

FIG. 3A is an example layout of an electrical circuit layer of an electrode patch assembly, in accordance with aspects of the present disclosure.

FIG. 3B a sectional view of the electrical circuit layer of FIG. 3A.

FIG. 3C another sectional view of the electrical circuit layer of FIG. 3A.

FIG. 3D is top view of a sled holder of an electrode patch assembly, in accordance with aspects of the present disclosure.

FIG. 3E is perspective view of the sled holder shown in FIG. 3D.

FIG. 3F is another perspective view of the sled holder shown in FIG. 3D.

FIG. 4A is front perspective view of a wireless monitoring system, in accordance with aspects of the present disclosure.

FIG. 4A is back perspective view of a wireless monitoring system, in accordance with aspects of the present disclosure.

FIG. 5A is a top view of an example electrode patch assembly, in accordance with aspects of the present disclosure.

FIG. 5B is a sectional view of the electrode patch assembly shown in FIG. 5A.

FIG. 6 are photographs showing an example electrode patch assembly, in accordance with aspects of the present disclosure.

FIG. 7A is a photograph showing an electrode patch assembly coupled to a subject's forehead, in accordance with aspects of the present disclosure.

FIG. 7B is another photograph showing a wireless monitoring system coupled to a subject's forehead, in accordance with aspects of the present disclosure.

FIG. 8 is an illustration showing a charging station for charging an electronics module, in accordance with aspects of the present disclosure.

FIG. 9 is a schematic showing modes of operation of a wireless monitoring system, in accordance with aspects of the present disclosure.

FIG. 10 is a schematic diagram of a flowchart setting forth steps of a process in accordance with aspects of the present disclosure

FIG. 11 are graphs comparing traditional EEG monitoring and monitoring using a system in accordance with aspects of the present disclosure.

### DETAILED DESCRIPTION OF THE INVENTION

The present disclosure provides a system and method for monitoring of physiological signals. As will become apparent from description below, the provided system and method have a wide range of applicability in various settings, including identifying, based on the measured physiological signals, such as electroencephalogram (“EEG”) signals, acute or long-term signatures indicative of a subject's medical condition. That is, the present platform may provide reliable monitoring over minutes, hours, and days, in operating rooms, intensive care units, and non-clinical settings. Identified signatures may then be used to determine a medical or brain condition of the subject, such as the onset and/or level of anesthesia, sedation, coma, sleep, pain, and others. In addition, the present system and method may also be used to determine an effectiveness of an administered treatment or medication.

The present monitoring platform includes an electrode patch assembly, which may refer broadly to any system, device or applicator having features and capabilities in accordance with the present disclosure. Non-limiting examples may include bandages, patches, headbands, wristbands, legbands, straps, necklaces, cuffs, belts, wearable electronics, and so forth, that are configured for placement and coupling to various portions of the body.

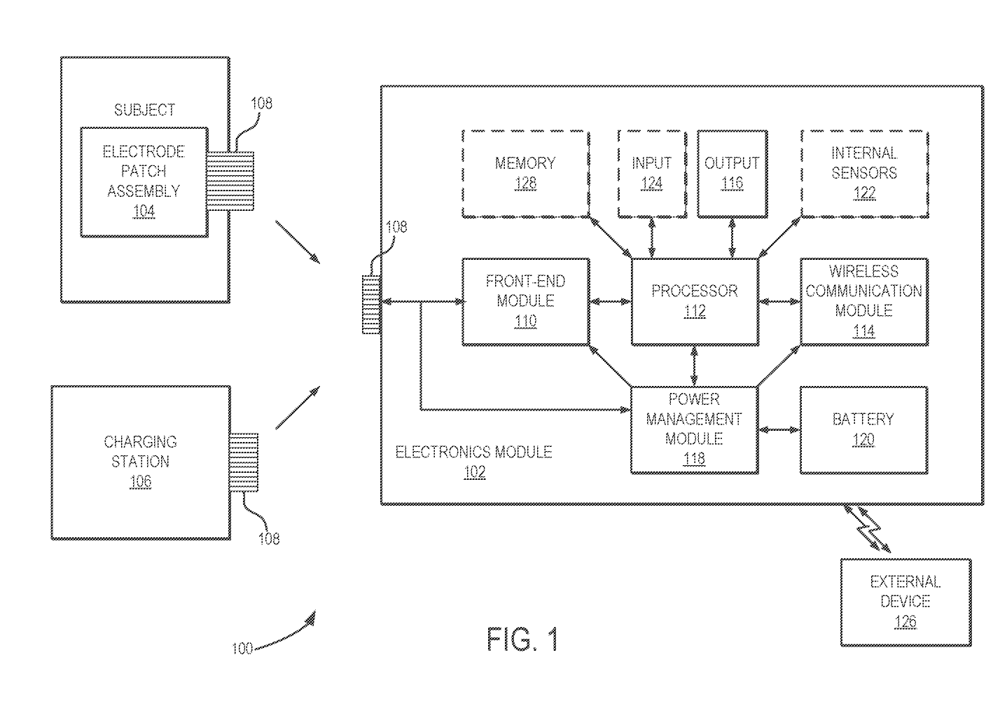

Referring particularly to FIG. 1, a schematic diagram of an example monitoring system 100, in accordance with aspects of the present disclosure, is shown. In some implementations, the monitoring system 100 may be a wireless monitoring system. As illustrated, the monitoring system 100 can include an electronics module 102, an electrode patch assembly 104, and a charging station 106, where the electrode patch assembly 104 and charging station 106 may be independently connectable by way of at least one electrical coupling 108, where the electrical coupling 108 includes a plurality of electrical contacts.

As shown in FIG. 1, the electronics module 102 may include a front-end module 110, at least one processor 112, a wireless communication module 114, and at least one output 116. The electronics module 102 may also include a power management module 118, a replaceable or rechargeable battery 120, and optionally a number of internal sensors 122, such as an accelerometer, and others. The electronics module 102 may also optionally include at least one input 124, and a memory 128 Elements of the electronics module 102 may be assembled using one or more printed circuit boards.

Specifically, the processor 112 may be configured to coordinate the various operation modes and states of the electronics module 102 using transitory and non-transitory instructions stored in a memory, as well as instructions provided via the wireless communication module 114 and/or input 122. As one non-limiting example, the processor 112 may include a low-power high performance microcontroller, including a microcontroller having self-programming flash memory, a boot code section, SRAM, EEPROM, an external bus interface, a multi-channel DMA controller, multi-channel event system, and other features.

In some aspects, the processor 112 may be configured to coordinate physiological signal acquisition and processing, wireless communication with an external device 126, power management, and other functions. By way of example, the external device 126 or remote host may be a computer, laptop, workstation, mobile device, tablet, phone, and so forth, configured to receive, process, and analyze received data, as well as transmit data and instructions. For example, the external device 126 may be configured to analyze acquired EEG data, and other physiological data, and determine a brain condition of the subject, such as the onset and/or level of anesthesia, sedation, coma, sleep, pain, and others. In addition, the external device 126 may be configured to determine an effectiveness of an administered treatment or medication based on the acquired physiological data.

In one implementation, the processor 112 may run a software application program that coordinates the acquisition and processing of detected physiological signals, such as EEG signals, electromyography (“EMG”) signals, galvanic skin response (“GSR”) signals, electrocardiogram (“ECG”) signals. and other physiological signals. In some aspects, the processor 112 may also be configured to acquire and process actigraphy or multi-axis accelerometer signals using the internal sensors 122, which may be used for detecting, for example, arousals in sleep or in the operating room, as well as a body position. In alternative embodiments, the processor 112 may obtain actigraphy signals from accelerometers included in the electrode patch assembly 104. Signal acquisition may be performed at a pre-determined sampling rate, or as instructed by the external device 126, and may depend upon the signal type.

The processor 112 may then set up a wired or wireless link to a host, receive commands and setup information, and transmit processed physiological signals in the form of digitized and/or compressed physiological data in real-time using a specific communication protocol, such as a wireless communication protocol. Alternatively, or additionally, the processor 112 may be configured to pre-process raw signal data, as well as store the pre-processed or raw signal data in the memory 128 for subsequent retrieval, pre-processing, and/or transmission. The processor 112 may also set up a battery charger integrated circuits, and display operational status using the output 116. In some aspects, the processor 112 may be configured to identify the type of electrode patch assembly 104 that is coupled to the electronics module 102. As such, modes of operation and operational parameters may be adapted based upon the detected and identified electrode patch assembly 104. For example, the processor 112 may determine the type of electrode patch assembly 104 by the number of types of signals received. In addition, the processor 112 may also be configured to detect whether a connection has been established or lost with the electrode patch assembly 104, and provide an indication to the user via the output 116.

Referring again to FIG. 1, the front-end module 110 of the electronics module 102 may be configured to receive and pre-process signals acquired using the electrode patch assembly 104, including filtering, amplifying, offsetting, and digitizing the acquired physiological signals. This allows for higher signal-to-noise ratio (“SNR”) data as well as efficient data manipulation and transmission. In one embodiment, the front-end module 110 may receive physiological signals from 8 separate channels. It may be readily appreciated that fewer or more channels may be utilized, depending upon the desired number of measurements, and measurement types.

As such, the front-end module 110 may include a wide variety of electrical components, including passive components, as well as an integrated circuit (“IC”) with amplifiers and an analog-to-digital (“A/D”) converter. In some embodiments, the front-end module 110 may include one or more low-pass, or high-pass filters with cutoff frequencies approximately between 0.5 and 500 Hz, although other values may be possible. The front-end module 110 may also include a band-pass filter, or any combinations of filters. In some aspects, the front-end module 110 alone, or in cooperation with the processor 112, may be configured to analyze analog or digitized physiological signals, or other measured signals, and coordinate an active noise cancellation process either via direct noise filtration of the measured signals, or via electric signals provided to shielding or grounding leads or layers in the electrode patch assembly 104, as will be described. In some aspects, the pre-processing may depend on signal characteristics, such as signal amplitudes, frequencies, phases, power spectra, noise profiles, and so forth of the acquired physiological signals. Also, in some aspects, such signal pre-processing may vary depending upon the identified electrode patch assembly 104, as described.

In one non-limiting example, the IC included in the front-end module 110 may be configured to provide one or more reference signals, in the form of bias voltage for instance, based on received EEG or other physiological signals signals, in order to remove unwanted voltage offsets from the received EEG or other signals. The offset EEG signals may then be amplified and converted into 24-bit digital values, for instance.

As mentioned, the processor 112 also communicates with a wireless communication module 114, allowing the wireless transmission and reception of data, instructions, and other information between the electronics module 102 and external device 126 via a customizable communication protocol. As such, the wireless communication module 114 includes a radio transceiver, and other hardware. For example, the radio transceiver may be a Bluetooth radio transceiver configured to manage both the physical and data link layers, although other types of wireless transceivers may be possible.

Referring again to FIG. 1, the electronics module 102 is also shown to include a power management module 118 that is configured to manage power distribution and use for the electronics module 102. In particular, the power management module 118 may be configured to provide appropriate power to the various components of the electronics module 102, as well as manage use and charging of the battery 120. As such, the power management module 118 may include a wide variety of electrical circuitry and components, including various ICs, linear voltage regulators, boost switching regulators, passive elements, and so forth.

For instance, voltage regulators in the power management module 118 may be used to convert a voltage of the battery 120 into lower voltages that are needed by the various components of the electronics module 102. Also, the power management module 118 may include a battery charger IC that can manage the proper charging of the battery 120. That is, when an external charge voltage provided by the charging station 106 is detected, by way of an electrical connection to the electrical coupling 108, the charger IC can apply a charge voltage and current to the battery 120 based on a current level of the battery 120, as detected by the power management module 118, for instance. As such, the charger IC can then manage charge voltage/current profile in order to safely charge the battery 120 and extend its life. In one example, the battery 120 may be a single cell (i.e. 3.6V) lithium-polymer rechargeable battery. The battery 120 may be configured and dimensioned in accordance with desired run time before recharging, and physical size.

As shown, the electronics module 102 includes an output 116, which may be in the form of LEDs, LCD displays, and the like, configured to provide various indications to a user. For instance, in some implementations, the output 116 may include one or more colored LEDs that indicate the operational state of the electronics module 102. By way of example, operational states of the electronics module 102 may include when the electronics module 102 is coupled to the charging station 106, when the battery 102 is charging, when the battery 120 is fully charged, when the battery 120 requires charging, when the electronics module 102 is connected to the electrode patch assembly 104, when data is being transmitted or received from the external device 126, when no connection is made to the external device 126, when a data connection is made to the external device 126 yet data is not being transmitted, and so forth. As shown in FIG. 1, the electronics module 102 may also include an input 124, in the form of buttons, switches, and other inputs. For example, the input 124 may include a power button, a reset button, toggle switches, and so forth.

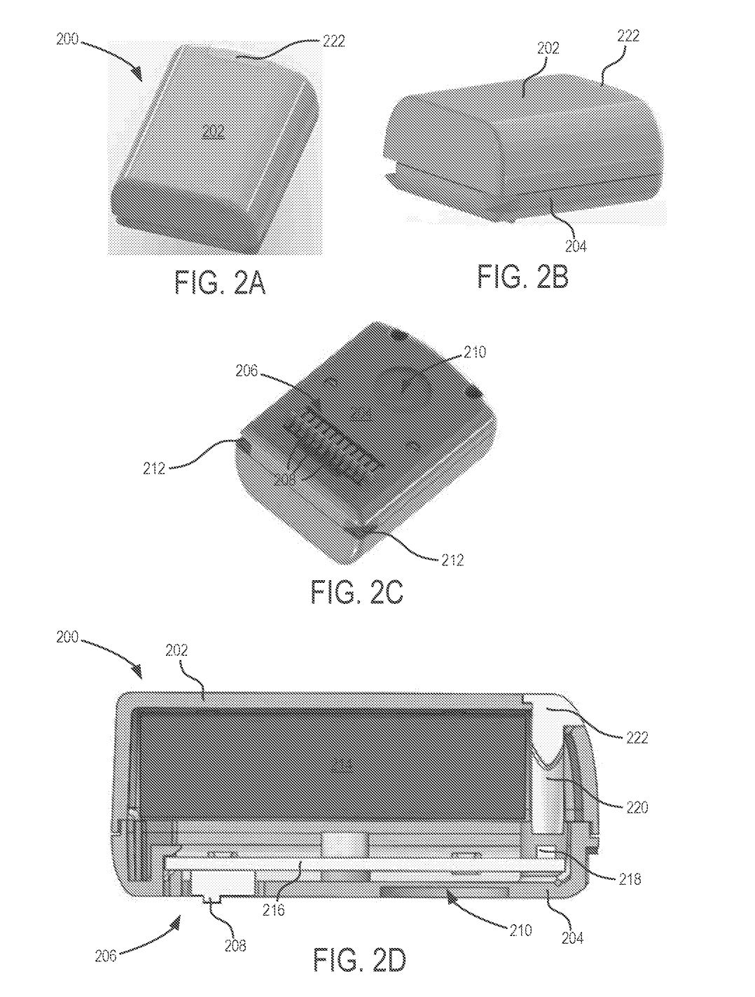

Turning now to FIGS. 2A-2D, one embodiment of an electronics module, as described with reference to FIG. 1, is shown. In particular, FIGS. 2A-2C show perspective views, while FIG. 2D shows a sectional view of the electronics module. As shown, the electronics module 200 includes a housing formed by a top portion 202 and bottom portion 204 that are coupled to one another using fasteners. In one example, the housing may be formed using a hard plastic or another suitable material. The top portion 202 and bottom portion 202 may be coupled together using various fasteners, such as screws, as shown in FIG. 2C, as well as other fasteners.

The bottom portion 204 of the housing includes an electrical coupling opening 206, which allows electrical contacts 208 to protrude therethrough (FIGS. 2C and 2D). As shown in the figures, the electrical contacts 208 may be mechanical contacts in the form of leaf-spring contacts that extend slightly beyond the outer surface of the bottom portion 204. Although it may be readily appreciated that other types of electrical contact types or connectors may also be possible, leaf-spring contacts are advantageous since they allow for a simpler electrical connection. In addition, when engaging with electrical pads on an electrode patch assembly, as will be described, the swiping motion of the leaf-spring contacts provides a self-cleaning of the contacts. Alternatively, pogo pin or spring-mounting pin contacts, or other mechanical contacts, may be used for the electrical contacts 208.

Referring again to FIG. 2C, the bottom portion 204 also includes a circular recess 210 that may be configured to hold a magnet (not shown). The magnet along with a counterpart magnet or metallic component of the electrode patch assembly, as will be described, allow the formation of a reproducible electrical connectivity between the leaf-spring contacts and contact pads configured on the electrode patch assembly. In this manner, a detachable yet reliable electrical connection can be made between the electrode patch assembly and electronics module 200, reducing signal interruption, contact resistance, and noise pickup. In addition, the snapping action of the magnet can give positive feedback to the user that the electronics module has been properly coupled. Furthermore, the bottom portion 204 also includes guidance slots 212 that facilitate engagement of the electronics module 200 with the electrode patch assembly.

Referring particularly to FIG. 2D, the inside of the housing is configured to hold a battery 214 and circuit board 216 that includes various electrical components. As described with reference to FIG. 1, in some implementations, the electronics module 200 can include one or more LEDs 218 for indicating various states of operation to a user. In order to make the light emitted by the LEDs visible to a user, the electronics module 200 also includes a light pipe 220. The light pipe 220 terminates into a diffuser 222 that is configured to direct the propagating light into multiple directions, thus allowing the states of operation indicated by the LEDs 218 to be seen from several angles. Although a single light pipe 220 is shown in FIGS. 2A-2D, it may be readily appreciated that design variations may include any number of light pipes, whose arrangement depend upon the particular circuit configuration.

Although the housing of the electronics module 200 is shown in a particular implementation in FIGS. 2A-2D, indeed various modification in shape, size, and features may be possible. In some aspects, it is preferable that the housing be configured to be as small as possible, for instance, just fitting the battery 214, circuit board 216 and light pipe 220 in FIG. 2D, to reduce its footprint and weight, and thereby increase patient comfort, allowing for long-term use.

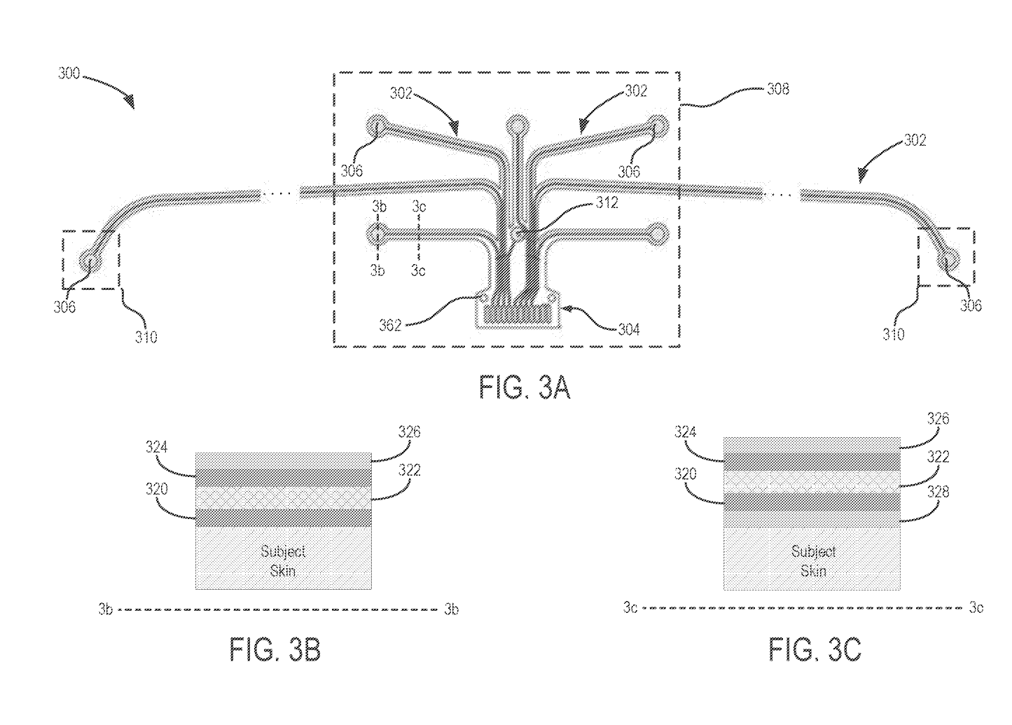

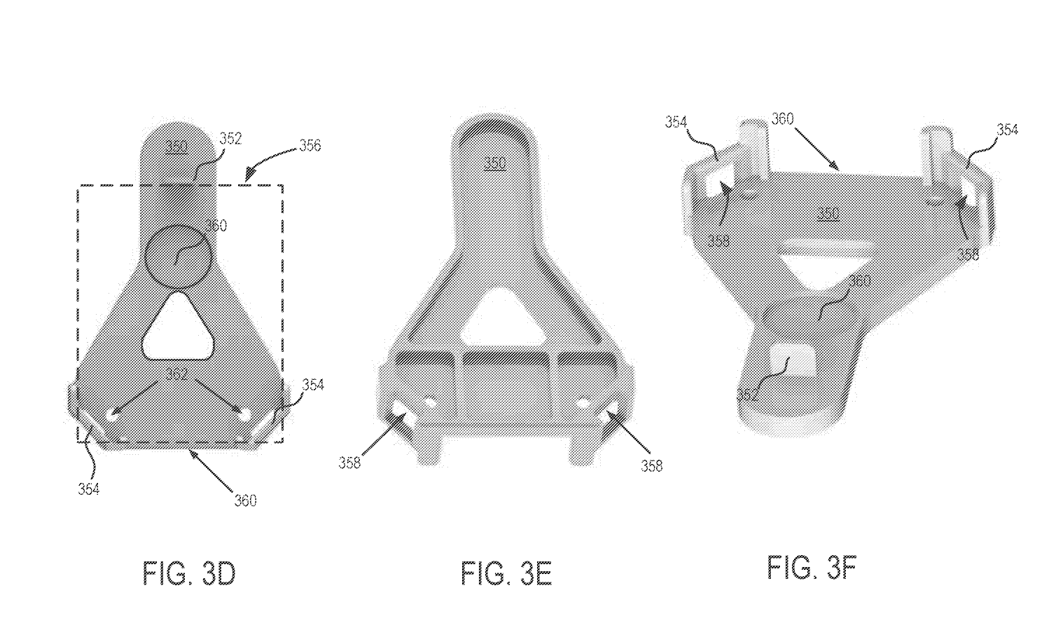

Referring now to FIGS. 3A-3F, one embodiment of an electrode patch assembly, as described with reference to FIG. 1, is shown. In particular, the electrode patch assembly includes a flexible circuit layer 300 (FIGS. 3A-3C), portions of which are configured to be coupled to a subject's skin using an adhesive layer, as will be described. The flexible electrode circuit 300 may be attached to a holder (FIGS. 3D-3F) that is configured to removably secure thereto an electronics module, as described with reference to FIGS. 1-2.

As shown in FIG. 3A, the flexible circuit layer 300 includes a plurality of electrical leads 302 originating in contact pads arranged on a contact area 304 and extending to separate electrodes 306. Each of the electrodes 306 may be configured to provide a low impedance electrical connection to a different location on the subject's skin, such as locations on the forehead, scalp, behind the ears, and others. In some implementations, the electrodes 306 may be coated with conductive gel to improve electrical contact with the subject's skin. As shown in the example of FIG. 3A, the flexible circuit layer 300 may include eight electrodes 306, with 6 of them being located in a central portion 308 of the flexible circuit layer 310, and 2 located in an extended portion 310 of the flexible circuit layer 310. It may be readily appreciated that any number of electrodes and electrode configurations may be included in the flexible circuit layer 300, depending upon the desired physiological signals to be measured, and their respective locations on the subject.

In one application, electrodes 306 included in the central portion 308 may be coupled to the subject's forehead, while electrodes 306 included in the extended portion 310 may be coupled to the mastoid processes (behind the ear) of the subject. For example, during one type of measurement, seven of the electrodes 306 may be used as EEG signal inputs and one as reference input, with the reference electrode 312 being located in the central portion 308 of the flexible circuit layer 300. Alternatively, various combinations of the electrodes can be used for additional reference schemes, for example, a common average reference or a Laplacian reference. In this configuration, various physiological signals may be obtained from the subject, including EEG alpha signals. In particular, this design facilitates the acquisition of occipital EEG alpha signals without the difficulties of using an area of head that has hair, or an area of the back of the head that includes large muscles producing interfering signals.

Referring specifically to FIGS. 3B and 3C, cross-sections of the flexible circuit layer 300 along lines 3b-3b and 3c-3c from FIG. 3A are shown. Specifically, FIG. 3B shows a cross-section along an example electrode 306 in the flexible circuit layer 300. The cross-section includes a conductive layer 320 in direct contact with the subject's skin, followed vertically by a base layer 322, a shielding layer 324 and a top insulating layer 326. Specifically the conductive layer 320 is configured to transmit electrical signals generated by subject, while the base layer 322 is configured to provide strength and flexibility to the flexible circuit layer 300. The shielding layer 324 is configured to provide protection against electromagnetic interference, and the top insulating layer 326 isolates the shielding layer 324 electrically. In some implementations, the base layer 322 may include a transparent or opaque polyester film or other suitable material, and the conductive layer 320 and/or shielding layer 324 may include a conductive ink or other conductive material, such as silver or silver chloride conductive ink.

FIG. 3C shows an example cross-section along an electrical lead 302, which a structure similar to FIG. 3B, with the addition of a bottom insulating layer 328 in direct contact with the subject's skin and the conductive layer 320. In this arrangement, the bottom insulating layer 328 prevents electrical contact with the subject along the length of the electrical lead 302, and ensures that the measured electrical signals are specific to the location of the electrode 306.

As described, the flexible electrode circuit 302 of FIGS. 3A-3C may be attached to holder that is configured to couple to and hold an electronics module. As such, FIGS. 3D-3F show an example “sled” holder 350 in accordance with aspects of the present disclosure. The holder 350 may include a plastic, or other suitable material, that is lightweight and provides sufficient structural rigidity. Referring specifically to FIG. 3D, the holder 350 may include a superior guidance post 352 and two inferior guidance posts 354 that define a space 356 for an electronics module, as described. In some designs, the superior guidance post 352 may include a small protrusion configured to engage with a small hole on the electronics module when properly positioned. In addition, the inferior guidance posts 354 may include rectangular openings 358 configured to engage a portion of electronics module, as shown in FIGS. 3E and 3F, and facilitate locking.

The holder 350 may also include a circular recess 356 configured to hold a magnetic or metallic material that is configured to engage with a magnet of an electronics module, as described with reference to FIG. 2C. When an electronics module is engaged with holder 350, the magnets, along with the guidance posts provide a secure yet removable connection. Although it may be appreciated that other locking mechanisms may also be utilized, features in the present design allow proper alignment of an electronics module, locking one end of the module in place as the other end is swung down and snapped into position, facilitated by the magnets of both the holder 350 and the electronics module.

The insertion and locking mechanism described above allows the electronics module to be easily and quickly attached while making a high quality electrical connection between the electronics module and the flexible electrode circuit due to the mechanical pivoting action that drags the leaf-spring contacts on the electronics module against the exposed conductive traces on the flexible material. The electronics module can then be easily removed by pulling the free end of the module until the strength of the bond between the magnets is broken. In this manner, multiple electronics modules can be removed and replaced with freshly charged ones without need for removing electrode patch assembly from the subject. This allows for monitoring for periods of time that are much longer than the battery life of any single electronics module with minimal interruption.

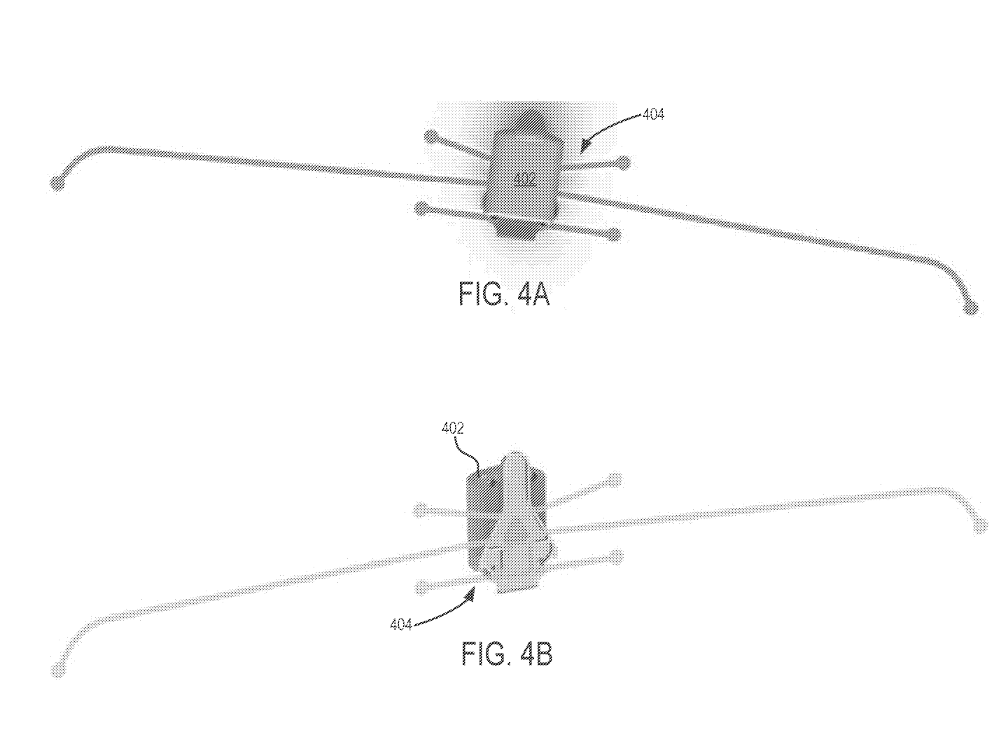

FIGS. 4A and 4B show different views of an electronics module 402 engaged with en electrode patch assembly 404, as described.

Referring again to FIG. 3A, the flexible circuit layer 300 may be attached to the holder 350 shown in FIGS. 3D-3F by way of an adhesive or adhesive-backed foam or tape. In particular, the contact area 304 of the flexible circuit layer 300 may wrap around an inferior portion 360 (FIG. 3D) of the holder 350 so that the leaf-spring contacts configured on an electronics module can make an electrical connection to the contact pads of the flexible electrode circuit 302. The contact pads on the contact area 304 may be alternatively replaced by counterparts to pogo pin or spring-mounting pin contacts, or other mechanical contacts. In some implementations, the holder 350, along with flexible circuit layer 300, can include two small holes 362 to facilitate proper positioning, as shown in FIGS. 3A and 3D, during a manufacturing process.

As mentioned, the flexible circuit layer 300 may be configured to be coupled to a subject's skin. As such, in some aspects, at least a portion of the flexible circuit layer 300 may also include an adhesive layer that is configured to provide attachment to the subject's skin (for clarity not shown in FIGS. 3A-3C). For example, the adhesive layer may be a thin, stretchable, breathable, adhesive coated membrane, such as 3M's Tegaderm material. This allows use over long periods of time without patient discomfort. In some implementations, the adhesive layer may cover a sufficient portion of the flexible circuit layer 300 to support the weight of the electrode patch assembly and electronics module, as well as adheres firm contact of the electrodes to the subject's skin.

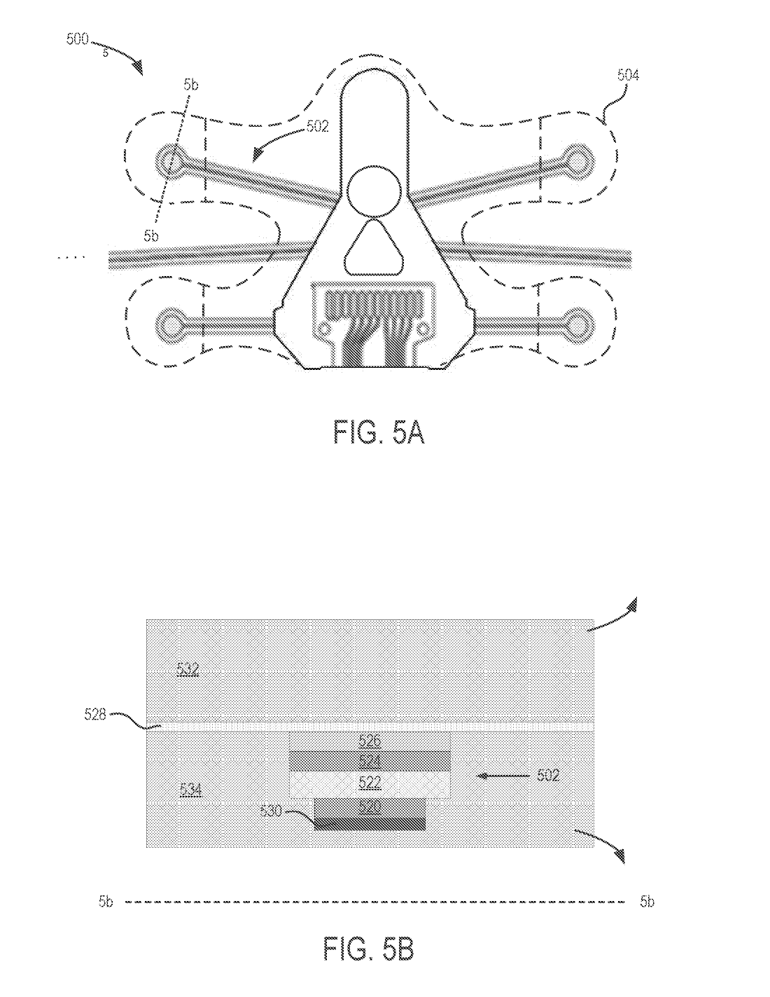

Prior to use, the adhesive layer may be protected by removable packaging. Referring now FIGS. 5A and 5B, one implementation of an electrode patch assembly 500 as packaged for distribution and storage is shown. In particular, FIG. 5A shows a top view of an example electrode patch assembly 500, depicting a flexible circuit layer 502 included in a packaging stack 504. The geometry of the packaging stack 504 is for illustration purposes only, and hence may vary in shape, dimension and coverage of the flexible circuit layer 502. An exterior packaging (not shown) may also surround the electrode patch assembly 500 keeping it sterile, clean, and ready for use.

FIG. 5B shows a cross-section along line 5b-5b of FIG. 5A, spanning an example electrode in the electrode patch assembly 500. The electrode includes a conductive layer 520, followed vertically by a base layer 522, a shielding layer 524 and a top insulating layer 526. The top insulating layer 526 is adjacent to an adhesive layer 528, as described. The conductive layer 520 may also include a gel layer 530, to facilitate electrical contact with a subject's skin. The entire structure is embedded between an application stiffener 532 on top, and a release liner 534 on the bottom.

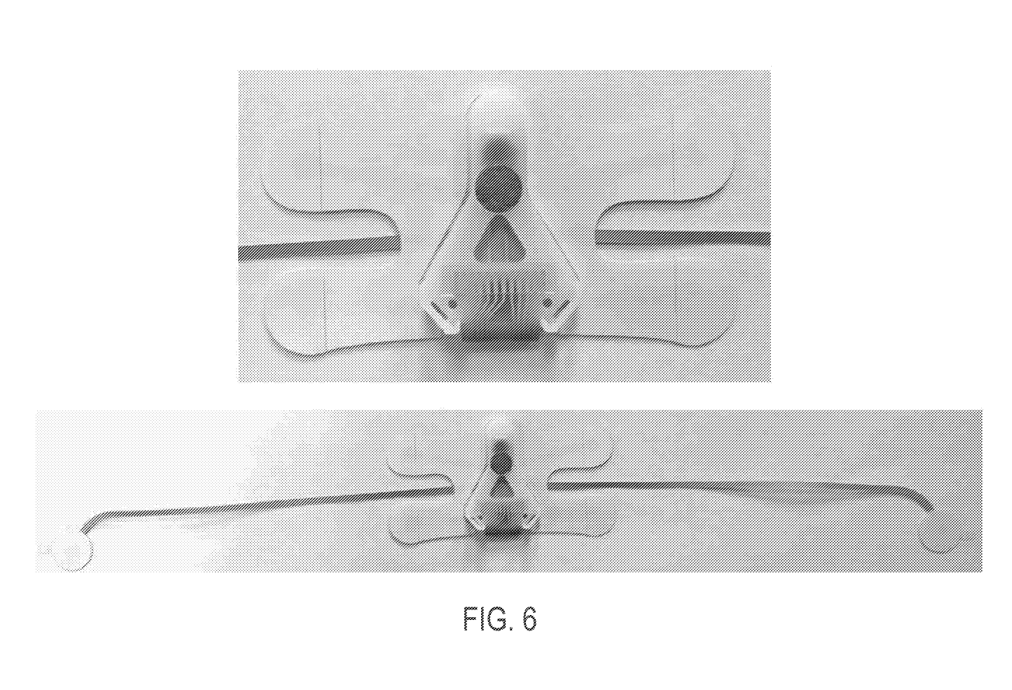

Before use, the user peels back and removes the release liner 534 from the bottom of the electrode patch assembly, exposing both the gel-covered conductive electrodes on the bottom side of the flexible circuit layer 502 as well as the adhesive layer 528. Conductive gel is kept in place over the exposed electrode and remains fresh due to the release liner 534 creating a tight seal to the flexible circuit layer 502. After the electrode patch assembly has been adhered to the skin, the application stiffener 532 may then be peeled off and removed, allowing the skin to breath freely through the adhesive layer 528. Views of an electrode patch assembly with an application stiffener in place and covering an adhesive layer are shown below in FIG. 6.

By keeping the conductive paths of electrical leads in the electrode patch assembly narrow, and by holding electrical leads and electrodes to the skin using a very thin, breathable, and stretchable adhesive layer, the present monitoring system is comfortable to wear for long periods of time. In addition, the described electrode patch assembly, while disposable, includes features that allow for improved clinical-grade physiological signal acquisition with high signal-to-noise ratio. Specifically, the conductive shield layer extending substantially over the length of the electrical leads in the electrode patch assembly and connecting to a reference point can reduce the coupling of external electro-magnetic signals to the signal paths. Also, a flexible, breathable adhesive layer holding the conductive paths tightly to the skin reduces motion artifacts, eliminating electrical voltages that are typically created when wires are able to move in free space. Other advantages of the present system include being small, lightweight, portable, and low-cost, allowing for simple use and being logistically efficient. In addition, physiological signal digitization close to the source, and optionally compression that allows for efficient and high quality data to be transmitted and analyzed.

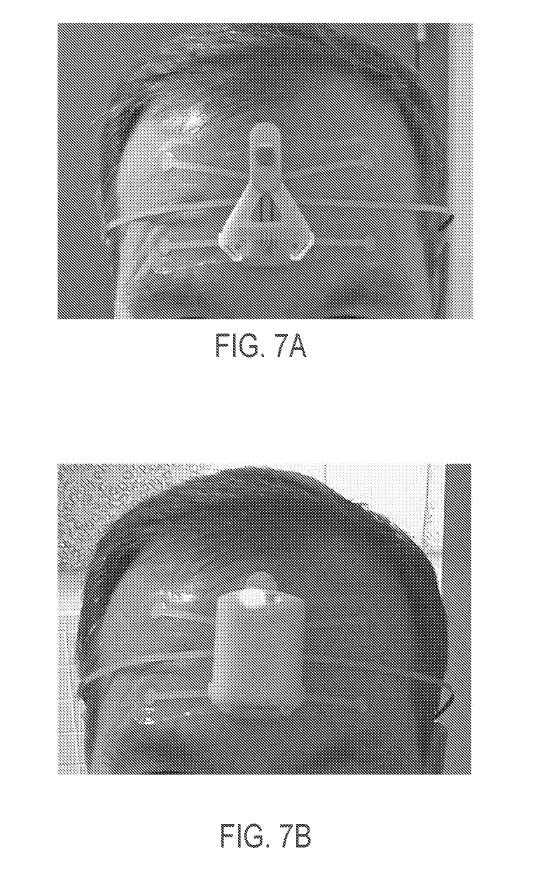

As an example, FIG. 7A shows a photograph of an electrode patch assembly, in accordance with aspects of the disclosure, attached to the forehead of a subject. FIG. 7B shows another photograph where an electronics module is coupled to an electrode patch assembly.

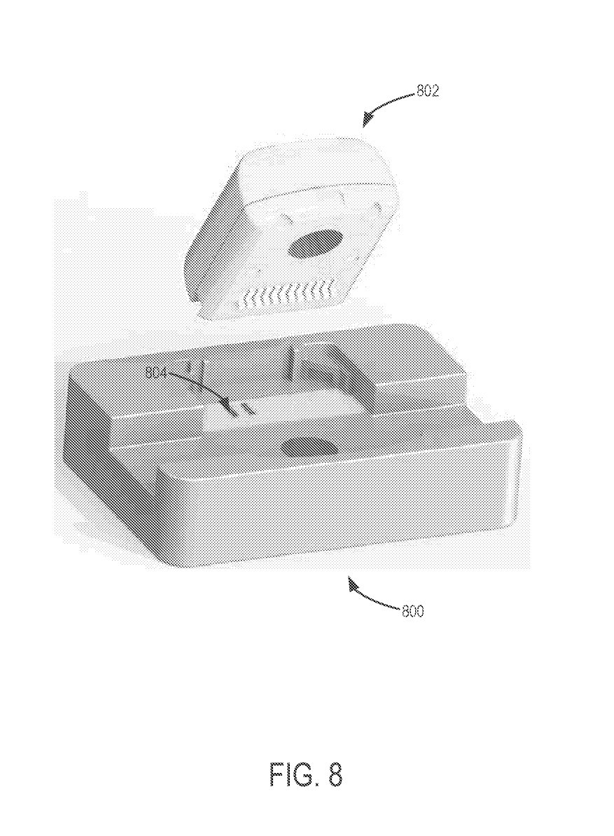

As described, electronics modules, in accordance with aspects of the present disclosure, may be recharged. FIG. 8 is a picture illustrating one embodiment of a charging station 800. As shown, the charging station 800 is configured to engage with the electronics module 802, and power is provided via input pins 802 that engage spring-leaf contacts on the electronics module 802. Using the same leaf-spring contacts alleviates the need for a secondary connector solely for the purpose of charging. As may be appreciated, the input pins 802 may very depending upon the contacts on the electronics module 802. For example, the input pins 802 may be counterparts to pogo pin or spring-mounting pin contacts. Although the charging station 800 is shown to engage with a single electronics module 802, it may be readily appreciated that modifications can be made to allow for multiple units to be charged. In some implementations, the charging station 800 may include LED or other outputs to indicate a charge status. Alternatively, outputs on the electronics module 802 may provide a charge or battery status indication. In addition, over-charge protection circuitry may be included in the charging station 800, allowing electronic modules to reside in the charging station 800 until they are required. Alternatively, such protection circuits may be built into the electronics module's charge circuitry.

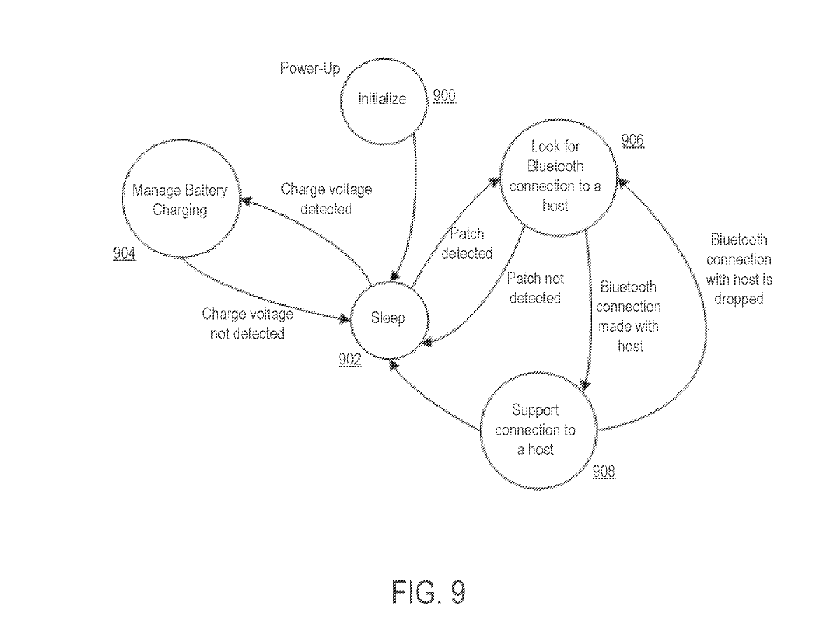

Referring now to FIG. 9, a schematic diagram illustrations modes operation of a system, in accordance with aspects of the present disclosure, is shown. As described a software application may run on a processor in the electronics module. The application may be configured to manage the state of the module, whereby certain operational settings are saved in non-volatile memory in order for these functional characteristics to survive the loss of power.

As indicated by step 900 in FIG. 9, when the electronics module is powered up, the application initializes the circuit and software variables. A front-end module may be set up to take measurements on specific channels, and the operation of the reference signal may established, all based on settings stored in non-volatile memory. If illegal values are found, default safe values may then be used instead, and written to non-volatile memory. When initialization is complete, the application may enter a sleep state, as indicated by step 902 in FIG. 9.

In the sleep state, the processor of the electronics module may turn off as much of the circuitry as possible in order to reduce power consumption. The processor may then be put into a low power sleep state, waking periodically to verify whether there is any reason to exit the sleep state. For instance, if a charge voltage is detected, the application enters the battery charging state, as indicated by step 904. In the battery charging state, the application may turn on the battery charger and monitor the charge state, displaying the charge status on the colored LED indicators, or other indicators, as appropriate. If the charge voltage disappears, the application may then return to the sleep state.

Alternatively, if an electrode patch is detected, a state that looks for Bluetooth connections may then be entered, as indicated by step 906. In addition, based upon the type of patch detected, the signal acquisition and/or signal processing may be adapted accordingly. Upon entering the state of looking for a Bluetooth connection to the host, the application may enable the Bluetooth radio transceiver and begin advertising the presence of the electronics module. If an electrode patch/patch assembly is no longer detected, the application may shut off the Bluetooth radio transceiver and return to the sleep state. If a data connection with a host is detected, advertising messages may be stopped and the state that supports the host may be entered, as indicated by step 908.

In the host connection support state, commands received from the host may be acted upon. The command for status may be answered, and the command for setting operational characteristics may be processed. If the command to begin streaming EEG and other physiological data is received, the front-end module may be enabled to begin capturing data at the pre-determined sampling rate. The application may then read samples and send them to the host in the pre-determined packet format. Sampling may continue until the command to stop is received, the Bluetooth link to the host is dropped, or the patch is no longer seen. When the electrical connection to the electrode patch is lost, the application may then return to the sleep state. If the host connection is dropped, the application may return to the state that looks for Bluetooth connections.

In some implementations, a wireless communication protocol between the electronics module and an external device, or host may include two communication modes, namely a command/response and streaming data. In the first communication mode, commands may be sent from the host to the electronics module, and the electronics module sends an immediate reply. The command sequence may include a fixed synch header, a command and supporting information, and a checksum. A checksum may be generated across the command and supporting information, allowing the electronics module to calculate the checksum on any command sequence that is received from the host and determine if the command sequence was received correctly. If not, the command sequence may be ignored. Responses to commands sent from the electronics module to the host may include a fixed synch header, the command that is being responded to, supporting information required to reply to the command, and a checksum. The checksum may be generated across the command and supporting information, allowing the host to calculate the checksum on any response sequence that is received from the electronics module and determine if the reply sequence was received correctly.

In the second communication mode, physiological data may be streamed continuously from the electronics module to the host without the need for any support from the host. The streaming packet format may include a fixed synch header, a sequence number, a block of physiological information, and a checksum. The checksum may generated over the entire block of physiological information, allowing the host to determine whether the physiological data was received correctly. Each packet of streaming physiological data increments the sequence number by one, indicating to the host when a block of physiological data was not received.

In some applications, physiological data may be natively acquired in a 24-bit format, yet the communication protocol may allow for the streaming of the data in a different format, such as 8-bit (upper 8 bits of the native 24-bit format), 16 bit (upper 16 bits), 24 bit (full native format data). In some aspects, the physiological data may be transformed into a 16-bit “delta” format. This last format compresses 24-bits of physiological data into 16 bits by subtracting the most recent 24-bit physiological value from the previous 24-bit value using 16-bit arithmetic. Compressed data may be advantageous for reducing power consumption, and thus increasing the time between battery replacement or recharging. As such, the format of the transmitted data may therefore depend upon the richness and fidelity of the data to be transmitted, energy needs and consumption of the system or device, as well as desired acquisition longevity or use. By way of example, one command may request the status and current operating modes of the electronics module, where the reply includes the firmware version, the number of samples in each physiological data streaming packet, the type of format used to send samples, the number of channels being sampled on the electrode patch, and the charge level of the battery. Another example command may allow the host to set the operating characteristics of the electronics module, including the number of the sampling rate of the physiological values, the number of physiological samples to include in each physiological data streaming packet, the format to use when streaming physiological data, the amplifier settings such as gain, the number of physiological channels to sample from the patch, and the referencing scheme. Yet another example command may turn physiological data streaming on or off. Yet another example command may request a patch type status response. Yet another command may set a gain setting. In addition, other commands may be used for diagnostic reasons to directly write and read the control registers within the front-end module of the electronics module.

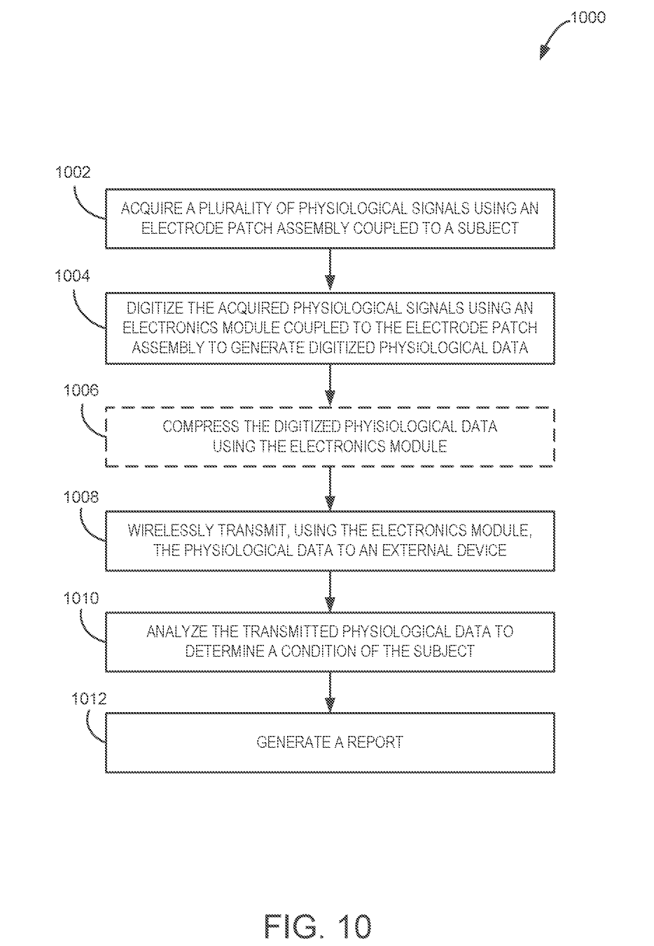

Turning now to FIG. 10, a flowchart setting forth steps of a process 1000 in accordance with aspects of the present disclosure is shown. The process 1000 may begin at process block 1002 with receiving a plurality of physiological signals, such as EEG signals, using an electrode patch assembly, as described. In some aspects, the acquisition may be adapted based on the type of electrode patch assembly utilized. Then at process block 1004, the acquired physiological signals may be digitized using and A/D converter included in an electronics module as described to generate digitized physiological data. Optionally, as indicated by process block 1006, the digitized physiological data may be compressed, before wireless transmission to an external device at process block 1008. As mentioned, this may include transmitting data in a “delta” format obtained by performing a subtraction of a higher-bit value using a lower-bit arithmetic. In some aspects, analog or digital data may be pre-processed, as well as stored or retrieved from a memory.

The transmitted physiological data may then be analyzed at process block 1010 to determine a condition of the subject. As described, this may include identifying specific signatures in the data that may then be used to determine a medical or brain condition of the subject, such as the onset and/or level of anesthesia, sedation, coma, sleep, pain, and others. In addition, an effectiveness of an administered treatment or medication may be determined based on the analyzed data. A report may then be generated, as indicated by process block 1012. The report may include real-time information, such as various waveforms representing measured physiological signals, as well as information or data derived therefrom.

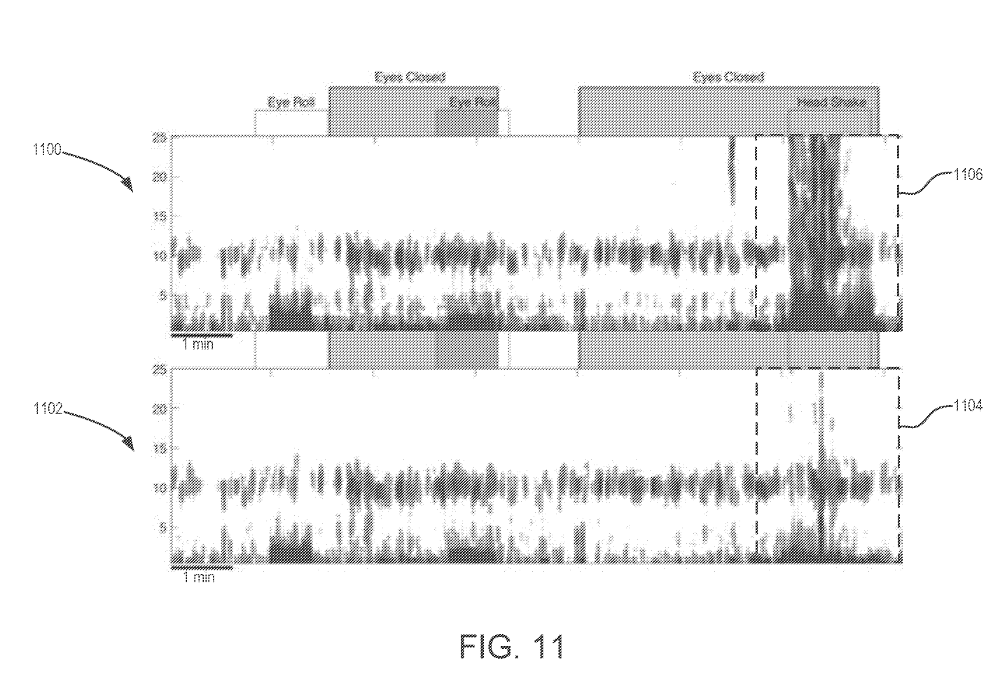

As may be appreciated from the above, the herein provided patient monitoring system and method affords significant advantages compared to current technologies. To further illustrate this point, a sleep experiment was carried out, where 10 minutes of EEG data was simultaneously recorded using a system, in accordance with the present disclosure, and a traditional sleep lab monitoring system. The results are shown in FIG. 11, with the top graph 1100 reflecting data obtained using the traditional system, while the bottom graph 1102 reflecting data obtained using the present system.

The results show similar closed-eye alpha (8-12 Hz) signals, and slow oscillation activity (<5 Hz), indicating that the present approach can provide clinical-quality data. However, unlike the traditional system, the present system is able to provide high-fidelity data even in problematic conditions, such as during patient motion, or head shaking, as indicated in FIG. 11. Specifically, a comparison of the data measured during the head shaking period reveals that the present approach, as seen in region 1104, results in significantly reduced artifacts compared to the traditional approach, as seen in region 1106.

Moreover, further experiments were performed with patients in a sleep lab setting, where simultaneous full-night sleep EEG recordings were acquired using the present approach and a standard 6-channel wired clinical EEG system. The recordings were scored independently by a clinical sleep technician using the procedures outlined by the American Academy of Sleep Medicine to determine the sleep stages across the night (the hypnogram). The results showed no significant difference between sleep stages when using the present system as compared to a traditional clinical system (Cohen's Kappa >0.6). This finding illustrates rigorous equivalence in the ability to capture brain activity during sleep using present approach and the gold standard of clinical systems used in sleep medicine.

The present invention has been described in terms of one or more preferred embodiments, and it should be appreciated that many equivalents, alternatives, variations, and modifications, aside from those expressly stated, are possible and within the scope of the invention.

1. An electroencephalogram (“EEG”) monitoring system comprising:
an electrode patch assembly configured to attach to a subject's skin, the electrode patch assembly comprising:
a flexible circuit layer having a plurality of electrical leads configured to acquire EEG signals from the subject, the flexible circuit layer having a shielding layer configured to substantially reduce a coupling of the plurality of electrical leads to external sources of noise, and
a holder to which the flexible circuit layer is secured;
an electronics module removably coupled to the holder and configured to engage electrical contacts on the flexible circuit layer, the electronics module comprising:
a front-end module configured to perform an active noise cancellation process on the acquired EEG signals and generate digitized data using noise cancelled signals;
a processor configured to transmit the digitized data using a wireless communication module; and
an external device configured to receive and analyze the digitized data transmitted to determine a condition of the subject.

2. The system of claim 1, wherein the electrode patch assembly is attached to the subject's skin using an adhesive layer contacting at least a portion of the flexible circuit layer.

3. The system of claim 1, wherein the electrode patch assembly is further configured to acquire at least one of electromyography (“EMG”) signals, galvanic skin response (“GSR”) signals, electrocardiogram (“ECG”) signals, actigraphy signals, or combinations thereof.

4. The system of claim 3, wherein the front-end module is further configured to perform the active noise cancellation process by applying a signal to the shielding layer based on a noise profile of the acquired EEG signals.

5. The system of claim 1, wherein the processor is further configured to analyze the digitized data to determine a type of the electrode patch assembly and adapt at least one of a signal acquisition and a signal processing based on the type determined.

6. The system of claim 5, wherein adapting the at least one of a signal acquisition and a signal processing includes modifying at least one of a gain and a filter.

7. The system of claim 1, wherein the electronics module is further configured to filter the acquired physiological signals using a low-pass filter, a high-pass filter, a band-pass filter, or combinations thereof.

8. The system of claim 1, wherein the processor is further configured to compress the digitized data prior to transmission.

9. The system of claim 1, wherein the external device is further configured to determine an onset or a level of anesthesia, sedation, coma, sleep, or pain, and generate a report.

10. The system of claim 1, wherein the external device is further configured to determine an effectiveness of an administered treatment or medication based the transmitted data analyzed and generate a report.

11. A system for wirelessly monitoring a subject, the system comprising:
an electrode patch assembly configured to attach to a subject's skin, the electrode patch assembly comprising:
a flexible circuit layer having a plurality of electrical leads configured to acquire physiological signals from the subject, the flexible circuit layer having a shielding layer configured to substantially reduce a coupling of the plurality of electrical leads to external sources of noise, and
a holder to which the flexible circuit layer is secured;
an electronics module removably coupled to the holder and configured to engage electrical contacts on the flexible circuit layer, the electronics module comprising:
a front-end module configured to generate digitized data using the acquired physiological signals;
a processor configured to wirelessly transmit, using a transceiver in the electronics module, the digitized data; and
an external device configured to communicate with the electronics module using a wireless communication protocol to receive the digitized data transmitted.

12. The system of claim 11, wherein the electrode patch assembly is attached to the subject's skin using an adhesive layer contacting at least a portion of the flexible circuit layer.

13. The system of claim 11, wherein the physiological signals comprise at least one of electroencephalogram (“EEG”) signals, electromyography (“EMG”) signals, galvanic skin response (“GSR”) signals, electrocardiogram (“ECG”) signals, actigraphy signals, or combinations thereof.

14. The system of claim 13, wherein the front-end module is further configured to perform an active noise cancellation process by applying a signal to the shielding layer based on a noise profile of the acquired physiological signals.

15. The system of claim 11, wherein the processor is further configured to analyze the digitized data determine a type of the electrode patch assembly and adapt at least one of a signal acquisition and a signal processing based on the type determined.

16. The system of claim 15, wherein adapting the at least one of a signal acquisition and a signal processing includes modifying at least one of a gain and a filter.

17. The system of claim 11, wherein the electronics module is further configured to filter the acquired physiological signals using a low-pass filter, a high-pass filter, a band-pass filter, or combinations thereof.

18. The system of claim 11, wherein the processor is further configured to compress the digitized data prior to transmission.

19. The system of claim 11, wherein the external device is further configured to analyze the digitized data transmitted to determine a condition of the subject and generate a report.

20. The system of claim 19, wherein the external device is further configured to determine an onset or a level of anesthesia, sedation, coma, sleep, or pain of the subject, or determine an effectiveness of an administered treatment or medication based the analysis.

21. The system of claim 11, wherein the electronics module is further configured to engage the electrical contacts on the flexible circuit layer using an electrical coupling having mechanical contacts.

22. A system for wirelessly monitoring a subject, the system comprising:
an electrode patch assembly configured to attach to a subject's skin, the electrode patch assembly comprising:
a flexible circuit layer having a plurality of electrical leads configured to acquire physiological signals from the subject, the flexible circuit layer having a shielding layer configured to substantially reduce a coupling of the plurality of electrical leads to external sources of noise, and
a holder to which the flexible circuit layer is secured;
an electronics module removably coupled to the holder and configured to engage electrical contacts on the flexible circuit layer using an electrical coupling, the electronics module comprising:
a front-end module configured to generate digitized data using the acquired physiological signals; and
a processor configured to compress and wirelessly transmit the digitized data, using a transceiver in the electronics module.

23. The system of claim 22, wherein the electrode patch assembly is attached to the subject's skin using an adhesive layer contacting at least a portion of the flexible circuit layer.

24. The system of claim 22, wherein the physiological signals comprise at least one of electroencephalogram (“EEG”) signals, electromyography (“EMG”) signals, galvanic skin response (“GSR”) signals, electrocardiogram (“ECG”) signals, actigraphy signals, or combinations thereof.

25. The system of claim 24, wherein the front-end module is further configured to perform an active noise cancellation process by applying a signal to the shielding layer based on a noise profile of the acquired physiological signals.

26. The system of claim 22, wherein the processor is further configured to analyze the digitized data determine a type of the electrode patch assembly and adapt at least one of a signal acquisition and a signal processing based on the type determined.

27. The system of claim 26, wherein adapting the at least one of a signal acquisition and a signal processing includes modifying at least one of a gain and a filter.

28. The system of claim 22, wherein the electronics module is further configured to filter the acquired physiological signals using a low-pass filter, a high-pass filter, a band-pass filter, or combinations thereof.

29. The system of claim 22, wherein the processor is further configured to compress the digitized data prior to transmission.

30. The system of claim 22, wherein the system further comprises configured to receive and analyze the digitized data transmitted to determine a condition of the subject and generate a report.

31. The system of claim 30, wherein the external device is further configured to determine an onset or a level of anesthesia, sedation, coma, sleep, or pain of the subject, or determine an effectiveness of an administered treatment or medication based the analysis.

32. The system of claim 22, wherein the electrical coupling further comprises mechanical contacts.
